(19) United States
(12) Patent Application Publication
(10) Pub. No.: US 2015/0191484 A1
(43) Pub. Date: Jul. 9, 2015

(54) NOVEL COMPOUNDS HAVING A TETRACYCLIC IRIDOID SKELTON AND AN ANTI-TRYPANOSOMAL AGENT COMPRISING THE SAME AS AN ACTIVE INGREDIENT

(71) Applicants: Shoji YAMAOKA, Tokyo (JP); Nobuo Ohta, Tokyo (JP); Mitsuko Suzuki, Tokyo (JP); Yukihiro Shoyama, Sasebo-shi (JP); Osamu Morinaga, Sasebo-shi (JP); Takuhiro Uto, Sasebo-shi (JP); Nguyen Huu Tung, Sasebo-shi (JP); Kwadwo Ansah Koram, Legon (GH); Kwabena Mante Bosompem, Legon (GH); Alexander Kwadwo Nyarko, Legon (GH); Daniel Adjei Boakye, Legon (GH); Irene Ayi, Legon (GH); Regina Appiah-Opong, Legon (GH); Dominic Adotei Edoh, Koforidua (GH); Laud Kenneth Nii-ayitey Okine, Accra (GH); Frederick Asare Aboagye, Larteh (GH)

(72) Inventors: Shoji YAMAOKA, Tokyo (JP); Nobuo Ohta, Tokyo (JP); Mitsuko Suzuki, Tokyo (JP); Yukihiro Shoyama, Sasebo-shi (JP); Osamu Morinaga, Sasebo-shi (JP); Takuhiro Uto, Sasebo-shi (JP); Nguyen Huu Tung, Sasebo-shi (JP); Kwadwo Ansah Koram, Legon (GH); Kwabena Mante Bosompem, Legon (GH); Alexander Kwadwo Nyarko, Legon (GH); Daniel Adjei Boakye, Legon (GH); Irene Ayi, Legon (GH); Regina Appiah-Opong, Legon (GH); Dominic Adotei Edoh, Koforidua (GH); Laud Kenneth Nii-ayitey Okine, Accra (GH); Frederick Asare Aboagye, Larteh (GH)

(73) Assignees: NATIONAL UNIVERSITY CORPORATION TOKYO MEDICAL AND DENTAL UNIVERSITY, Tokyo (JP); CENTER FOR SCIENTIFIC RESEARCH INTO PLANT MEDICINE, Mampong-Akuapem (GH); UNIVERSITY OF GHANA (NOGUCHI MEMORIAL INSTITUTE FOR MEDICAL RESEARCH), Legon (GH); NAGASAKI INTERNATIONAL UNIVERSITY, Sasebo-shi (JP)

(21) Appl. No.: 14/149,262

(22) Filed: Jan. 7, 2014

(51) Int. Cl. C07D 493/16

(52) U.S. Cl. CPC C07D 493/16

(57) ABSTRACT

The present invention provides anti-trypanosomal agent for treating, preventing Trypanosomiasis of mammals, which comprises a compound having the tetracyclic iridoid skeleton represented by a general formula (I).

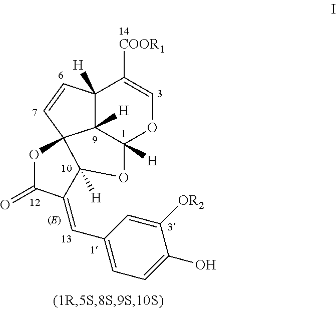

### TECHNICAL FIELD OF THE INVENTION

The present invention relates to novel compounds having a tetracyclic iridoid skeleton which have anti-trypanosomal activity, and an anti-trypanosomal agent comprising the compound.

### BACKGROUND ART

Trypanosomiasis is a collective designation of infectious disease common to man and mammals caused by a protozoa belonging to the genus Trypanosoma. Trypanosoma protozoa is infectious for many hosts including humans and domestic animals, and causes Trypanosomiasis such as African Trypanosomiasis called as sleeping sickness and American Trypanosomiasis called as Chagas' disease. The African Trypanosomiasis is caused by Trypanosoma transmitted through tsetse flies. In a patient of the Trypanosomiasis, the protozoa appear in the blood in about 10 days after infection. The protozoa grow in the blood and cause fever, physical weakness, headache, pain of muscles and joints in the early stage. The central nervous system is affected to show symptoms such as mental confusion and systemic convulsion, and finally the patients lapse into lethargy and ultimately die in a chronic stage.

It is said that there are 200,000 to 300,000 new Trypanosomiasis patients in Africa every year. Furthermore, Trypanosomiasis of domestic animals as nagana caused by Trypanosoma is serious. Several hundred thousands of cattle which are to be protein sources for people die every year in Africa.

In African Trypanosomiasis, Trypanosoma brucei brucei causes nagana of domestic animals, and Trypanosoma brucei rhodesiense, and Trypanosoma brucei gambiense cause sleeping sickness of man. American Trypanosomiasis is caused by Trypanosoma cruzi.

Vaccines against Trypanosomiasis have not been developed. For the treatment of Trypanosomiasis, Suramin, Pentamidine Isethionate (Sun T, Zhang Y, Nucleic Acids Research 2008, 36(5):1654-1664; Barrett M P et al., British Journal of Pharmacology 2007, 152(8):1155-1171; Wang C C, Annual Review of Pharmacology and Toxicology 1995, 35(11):93-127), Melarsoprol, Melarsonyl Potassium, Nitrofurazone and the like are used. However, these drugs have side effects such as cytotoxicity on mammalian cells. Accordingly, novel anti-trypanosomal agents without side effects have been desired.

It was reported that some compounds isolated from Morinda lucida had anti-protozoa activity. For example, it was reported that anthraquinones isolated from Morinda lucida has antileishmanial and antimalarial activities (A. Sittie et al., Planta Med. 65 (1999), pp. 259-). Furthermore, it was reported that Oruwacin isolated from Morinda lucida has antileishmanial activity (E. Kayode Adesogan, Phytochemistry, 1979, Vol. 18, pp. 175-176; P. J. Stephens, J. Nat. Prod., 2008, Vol. 71, pp. 285-288).

### BRIEF DESCRIPTION OF THE DRAWINGS

FIG. 1 shows the structure of tetracyclic spirolactone iridoids (2: ML-2-2 and 3: ML-2-3, Oruwalol (1), Ursolic acid (4) and Oleanolic acid (5) isolated from leaves of M. lucida.

FIG. 2 shows the protocol for fractionating and isolating the compounds.

FIG. 3 shows anti-trypanosomal activity (IC₅₀ value) of ML-2-2, ML-2-3, Oruwalol, Ursolic acid and Oleanolic acid.

FIG. 4 shows the results of Nexin assay for apoptosis using Trypanosoma cells incubated with ML-2-2 or ML-2-3. N.C. stands for negative control: Trypanosoma cells incubated with dimethyl sulfoxide (DMSO) alone.

FIG. 5 shows proportions of early and late stages of apoptotic cells among all viable cells at different incubation times.

FIG. 6 shows induction of apoptosis by ML-2-3 (5 μM) in viable Trypanosoma cells confirmed by FACS analysis using Multi-caspase assay. N.C. stands for negative control: Trypanosoma cells incubated with DMSO alone.

FIG. 7 shows histograms of cell cycle in Trypanosoma cells treated with either ML-2-2 or ML-2-3, indicating reduction in G2/M phase cells and increase in sub-G1 phase cells in ML-2-3 treated cells. N.C. stands for negative control: Trypanosoma cells incubated with DMSO alone.

FIG. 8 shows nuclear fragmentation and suppression of α-tubulin expression with resultant no flagellum phenotype. N.C. stands for negative control: Trypanosoma cells incubated with DMSO alone.

### SUMMARY OF THE INVENTION

The present invention is directed to a therapeutic composition for treating or preventing Trypanosomiasis.

The present inventors carried out screening of anti-trypanosomal activity for approximately 100 pharmaceutical plants grown in Ghana. The present inventors found that Morinda lucida had strong anti-trypanosomal activity. Four active ingredients including two novel compounds were purified and chemically identified. Two novel active compounds had a tetracyclic iridoid skeleton. They were called as ML-2-2 and ML-2-3, respectively. The structure of ML-2-2 is different from that of ML-2-3 in that ML-2-3 has COOH group at position 4 whereas ML-2-2 has COOCH₃ at position 4. Although ML-2-2 is an ester, ML-2-3 is a carboxylic acid.

The present inventors studied the anti-trypanosomal activity and cytotoxicity of ML-2-2 and ML-2-3, and found that ML-2-3 had strong anti-trypanosomal activity and had little cytotoxicity on mammalian cells whereas ML-2-2 had strong anti-trypanosomal activity and relatively higher cytotoxicity on mammalian cells. Thus, the present inventors have completed the present invention.

Specifically, the present invention is as follows.

[1] A compound having the tetracyclic iridoid skeleton represented by a general formula (I).

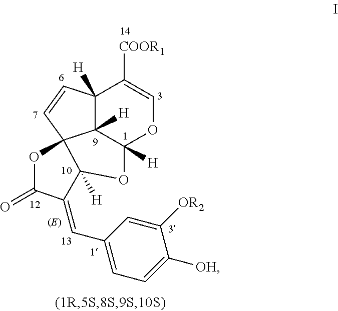

wherein R₁ and R₂ are independently, hydrogen or C₁-C₈ alkyl.
[2] The compound according to [1], which is isolated from Morinda lucida.
[3] The compound according to [1], which is represented by a formula (II).

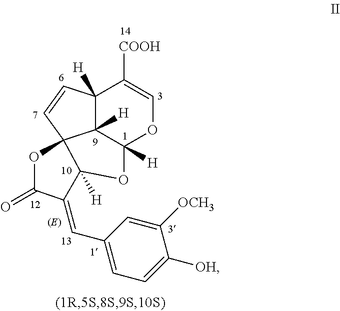

[4] The compound according to [1], which is represented by a formula (III).

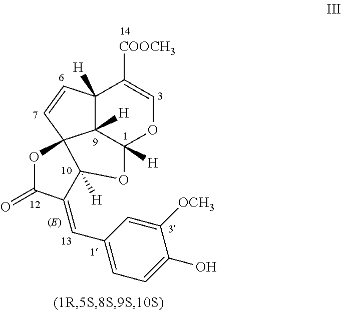

[5] An anti-trypanosomal agent for preventing or treating Trypanosomiasis comprising the compound according to [1] as an active ingredient.
[6] The anti-trypanosomal agent according to [5], which comprises the compound according to [3] as an active ingredient.
[7] The anti-trypanosomal agent according to [5], which comprises the compound according to [4] as an active ingredient.
[8] The anti-trypanosomal agent according to [5], wherein the Trypanosomiasis is African Trypanosomiasis or American Trypanosomiasis.
[9] The anti-trypanosomal agent according to [5], which has low cytotoxicity on mammalian cells.
[10] The anti-trypanosomal agent according to [5], wherein the selectivity index (SI) of the compound on normal human fibroblast is more than 8.
[11] A method for preventing or treating Trypanosomiasis, which comprises administering the compound according to [1] to a mammal.
[12] The method for preventing or treating Trypanosomiasis according to [11], which comprises administering the compound according to [3] to a mammal.
[13] The method for preventing or treating Trypanosomiasis according to [11], which comprises administering the compound according to [4] to a mammal.
[14] The method according to [11], wherein the Trypanosomiasis is African Trypanosomiasis or American Trypanosomiasis.
[15] The method according to [11], wherein the compound has low cytotoxicity on mammalian cells.
[16] The method according to [11], wherein the selectivity index (SI) of the compound on normal human fibroblast is more than 8.
[17] The method according to [11], wherein the subject is a domestic animal.
[18] The method according to [11], wherein the subject is a human.

### DETAILED DESCRIPTION OF THE PRESENT INVENTION

The compound having a tetracyclic iridoid skeleton of the present invention is represented by a general formula I.

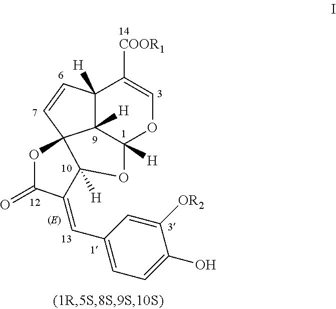

wherein R₁ and R₂ are independently, hydrogen or C₁-C₈ alkyl. The C₁-C₈ alkyl is preferably methyl (CH₃), ethyl (C₂H₅), propyl (C₃H₇), isopropyl ((CH₃)₂CH), butyl (C₄H₉), pentyl (C₅H₁₁) or hexyl (C₆H₁₃).

The compound having the tetracyclic iridoid skeleton has five chiral carbon atoms at positions 1, 5, 8, 9 and 10. The stereochemistry of the compound is (1R, 5S, 8S, 9S, 10S).

An example of the compound is a compound represented by the formula II.

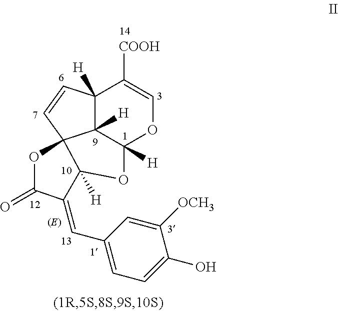

The compound is tetracyclic spirolactone iridoids in which R₁ is H and R₂ is CH₃.

The compound is called as ML-2-3 in the present invention. The general name of ML-2-3 is (3aS,4aR,4a¹S,7aS,9aS,3E)-3-(4-hydroxy-3-methoxybenzylidene)-2-oxo-2,3,3a,4a,4a¹,7a-hexahydro-1,4,5-trioxadicyclopenta[a,hi]indene-7-carboxylic acid.

Another example of the compound is a compound represented by the formula III.

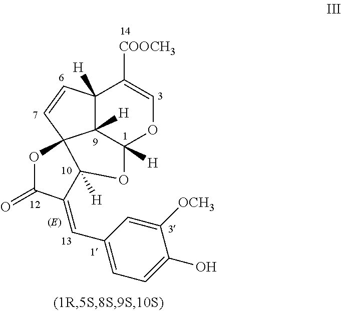

The compound is tetracyclic spirolactone iridoids in which R₁ is CH₃ and R₂ is CH₃. The general name of ML-2-2 is (3aS,4aR,4a¹S,7aS,9aS,3E)-methyl 3-(4-hydroxy-3-methoxybenzylidene)-2-oxo-2,3,3a,4a,4a¹,7a-hexahydro-1,4,5-trioxadicyclopenta[a,hi]indene-7-carboxylate. Although ML-2-2 is an ester, ML-2-3 is a carboxylic acid.

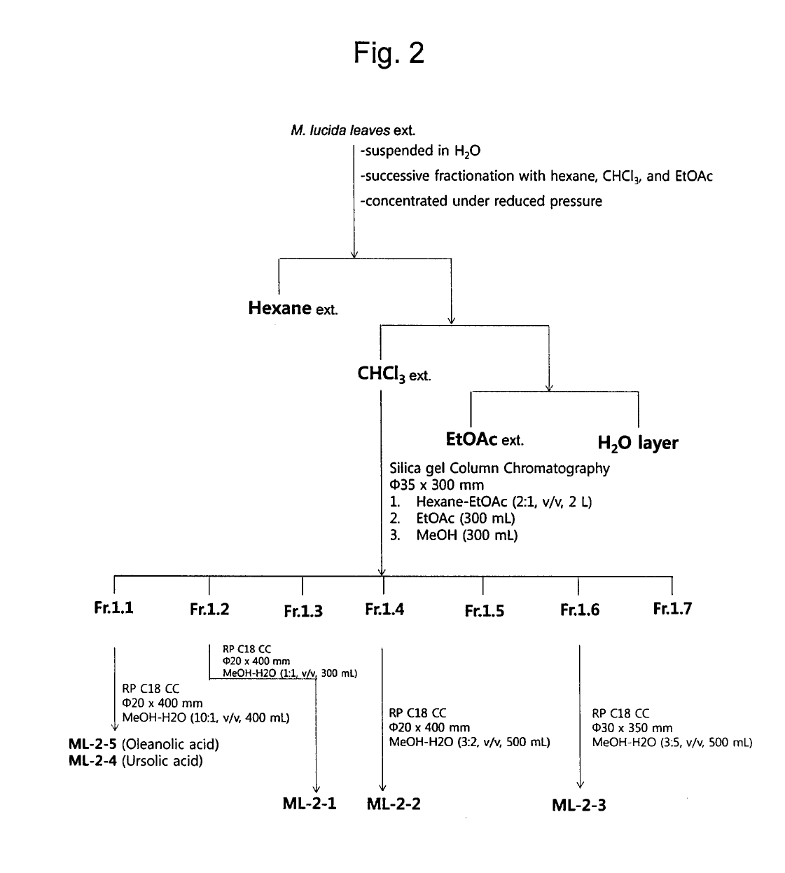

The compound represented by the general formula I is isolated from a plant belonging to genus Morinda which grows wild in most of the Central and West Africa. Specifically, the compound can be isolated from Morinda lucida. The compound can be isolated from any part of Morinda lucida. Leaves of Morinda lucida are preferable as source of the compound. For example, ML-2-2 and ML-2-3 are isolated from Morinda lucida as follows: Dried leaves of Morinda lucida are treated with aqueous ethanol to obtain a crude extract. The crude extract is then successively partitioned with hexane, CHCl₃ and EtOAc to obtain soluble fraction of each. The CHCl₃ fraction was subjected to a silica gel column with hexane-EtOAc at the mobile phase to obtain sub-fractions. The fraction having high anti-trypanosomal activity is obtained and further chromatographed over a reversed-phase (RP) column with MeOH—H₂O to obtain ML-2-3 fraction and ML-2-2 fraction. The exemplified protocol to isolate ML-2-3 fraction and ML-2-2 fraction is shown by FIG. 2. In the process of isolating ML-2-3 and ML-2-2, extract or fraction are tested for the anti-trypanosomal activity at each stage. The anti-trypanosomal activity can be determined by adding a candidate fraction or compound to in vitro Trypanosoma cells. Trypanosoma parasites are single-celled organisms. In the present invention, the term “Trypanosoma cells” means “Trypanosoma parasites”. Furthermore, in the present invention, the anti-trypanosomal activity is also referred to as trypanocidal activity.

The structural determination of the compound having a tetracyclic iridoid skeleton of the present invention can be achieved with NMR (nuclear magnetic resonance analysis).

The compound represented by the general formula I has strong anti-trypanosomal activity. Especially, ML-2-3 has strong anti-trypanosomal activity. In particular, ML-2-3 inhibits the expression of α-tubulin and inhibits flagella formation of Trypanosoma cells, and induces apoptosis in Trypanosoma cells. ML-2-3 induces both early and late stages apoptosis. Treatment of Trypanosoma cells with ML-2-3 significantly decreases the numbers of cells in G2 and M phase during cell cycle. Furthermore, DNA fragmentation that occurs during apoptosis is observed in the Trypanosoma cells.

ML2-2 has also anti-trypanosomal activity. The anti-trypanosomal activity of ML-2-2 is stronger than that of ML-2-3. However, ML-2-2 does not induce apoptosis in Trypanosoma cells, and does not cause any alteration during cell cycle.

Morinda lucida includes another compound called Oruwacin which is an enantiomer of ML-2-2. Oruwacin is represented by a formula IV.

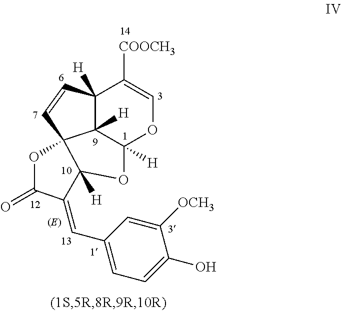

It was reported that Oruwacin has anti-Leishmania activity (E. Kayode Adesogan, Phytochemistry, 1979, Vol. 18, pp. 175-176; P. J. Stephens, J. Nat. Prod., 2008, Vol. 71, pp. 285-288). It has been known that enantiomers have different functions. Accordingly, the anti-Leishmania activity of Oruwacin does not predict the anti-trypanosomal activity of ML-2-2. Of course, the anti-trypanosomal activity of ML-2-3 and ML-2-2 is not predicted at all.

The important difference between ML-2-3 and ML-2-2 is that ML-2-2 has relatively higher cytotoxicity on mammalian cells whereas ML-2-3 does not have problematic cytotoxicity on mammalian cells. Much as ML-2-3 can be preferably used as a therapeutic agent for Trypanosomiasis of mammals, ML-2-2 can also be used as a therapeutic agent for mammals.

ML-2-3 has low cytotoxicity on mammalian cells. The cytotoxicity of ML-2-3 is very low compared to the cytotoxicity of ML-2-2. The cytotoxicity of ML-2-2 and ML-2-3 on mammalian cells can be compared as IC₅₀ value. The usefulness of the compounds for a therapeutic agent for mammals including humans can be compared by a selectivity index (SI). For a compound to be accepted as a useful therapeutic agent for mammals means that the compound should have high anti-trypanosomal activity but low cytotoxicity on mammals. The selectivity index (SI) is represented by an equation: IC₅₀ of cytotoxicity on mammalian cells/IC₅₀ of anti-trypanosomal activity. In particular, it is represented by an equation: IC₅₀ against human cells/IC₅₀ against Trypanosoma such as Trypanosoma brucei. For example, IC₅₀ (μM) of ML-2-3 for 48 hours against various human cells are about 2 times to more than 100 times higher than that of ML-2-2. IC₅₀ (μM) of ML-2-3 against HF-19 and HCT-15 is more than 50, which means that ML2-3 is non-toxic at the highest assayed dose in HF-19 and HCT-15. IC₅₀ (μM) of ML-2-2 against Trypanosoma cells is about 1 to 1.5 whereas that of ML-2-3 is about 3 to 5, preferably about 3.5 to 4. The selectivity index (SI) of ML-2-2 is about 0.09 to 12.63 whereas the selectivity index (SI) of ML-2-3 is about 3.2 to more than 13. The IC₅₀ and selectivity index means that ML-2-3 has low cytotoxicity on human cells and high anti-trypanosomal activity. The anti-trypanosomal activity of ML-2-3 is very selective and potent. Considering the reasonable level of the selectivity index (SI), ML-2-2 is also useful as anti-trypanosomal agent.

The present invention includes anti-trypanosomal agent comprising the compound having a tetracyclic iridoid skeleton which is represented by the general formula I as an active ingredient. The anti-trypanosomal agent can be used to prevent or treat Trypanosomiasis. It can also be used to prevent the infection of Trypanosoma to mammals. Further, it can be used to inhibit the growth of Trypanosoma in mammals.

The Trypanosomiasis includes any infectious disease caused by the infection of any Trypanosoma protozoa. It includes African Trypanosomiasis and American Trypanosomiasis. The Trypanosoma protozoa which causes the Trypanosomiasis includes Trypanosoma brucei brucei, Trypanosoma brucei rhodesiense, Trypanosoma brucei gambiense, Trypanosoma evansi, Trypanosoma congolense, Trypanosoma vivax, Trypanosoma hippicum and the like. Trypanosoma brucei brucei, Trypanosoma congolense or Trypanosoma vivax cause nagana of domestic animals, and Trypanosoma brucei rhodesiense, and Trypanosoma brucei gambiense causes sleeping sickness of man. American Trypanosomiasis is caused by Trypanosoma cruzi.

Any mammals which are known to suffer from Trypanosomiasis can be treated or any mammals can be prevented from the infection of Trypanosoma using the compound having a tetracyclic iridoid skeleton which is represented by the general formula I. The mammals include humans, cattle, horse, pig and the like.

Habitats of Trypanosoma protozoa include blood, spinal fluid and the like. The preferable habitat is blood.

The composition may include other ingredients such as a pharmacologically acceptable carrier, diluent or excipient. The pharmaceutical composition of the present invention can be administered in various forms. Examples of such an administration form include oral administration using tablets, capsules, granules, powders or syrups, or parenteral administration using injection, drop or suppository. Such a composition is produced by any known method and comprises a carrier, a diluent and an excipient, which are commonly used in the pharmaceutical field. For example, as a carrier or excipient used for a tablet, lactose, magnesium stearate or the like is used. An injection is prepared by dissolving, suspending or emulsifying the compound of the present invention or a salt thereof in a sterile aqueous or oily solution. Examples of aqueous solution used for an injection include a physiological salt solution and an isotonic solution containing glucose or another adjuvant, and the aqueous solution may be used in combination with an appropriate solution adjuvant such as alcohol, polyalcohol such as propylene glycol or a nonionic surfactant. Examples of the above-mentioned oily solution include sesame oil, soybean oil and so on, and the oily solution may be used in combination with a solution adjuvant such as benzyl benzoate or benzyl alcohol.

The dosage applied depends on symptom, age, body weight and others. In the case of oral administration, generally, it is approximately a range of 0.001 mg to 1,000 mg per kg body weight per day, and the compound with the above dosage is administered all at once, or divided several times throughout a day. In contrast, in the case of parenteral administration, 0.001 mg to 1,000 mg of the compound is administered per kg body weight per day in the form of a subcutaneous injection, intramuscular injection or intravenous injection.

The compound having a tetracyclic iridoid skeleton which is represented by the general formula I can be used as an excellent lead compound against Trypanosoma

The present invention also provides a method of preventing or treating Trypanosomiasis. The method comprises administering the therapeutically effective amount of the compound having a tetracyclic iridoid skeleton which is represented by the general formula I in a mammal suffering from Trypanosomiasis or a mammal which has risk to be infected with Trypanosoma such as a mammal which lives in an area where Trypanosoma is present (or thrives).

The present invention also provides the compound having a tetracyclic iridoid skeleton which is represented by the general formula I for use in the prevention or treatment of Trypanosomiasis. The present invention further provides use of the compound having a tetracyclic iridoid skeleton which is represented by the general formula I in the manufacture of anti-trypanosomal agent.

### EXAMPLE

### Isolation of Anti-Trypanosomal Compound from Morinda lucida and the Evaluation of the Compound for the Anti-Trypanosomal Activity

### Methods

### Reconstitution of Plant Materials

### Crude Extract

Ten milligrams of Morinda lucida crude extract was weighed into a 1.5 ml Eppendorf tube and 1 ml of 50% ethanol solution was added. The solution was vortexed to get a 10 mg/ml stock homogenous solution. The stock solution was diluted with HMI-9 culture medium (based on IMDM supplemented with certain ingredients such as salts and amino acids and 5% FBS, Yabu et al, 1998) to a concentration of 400 μg/ml inside a clean bench. The solution was filtered into new sterile tubes.

### Fractions and Compounds

Ten milligrams of each fraction/compound of Morinda lucida was weighed into a 1.5 ml Eppendorf tube and 100 μl of 100% DMSO was added to get a stock concentration of 100 mg/ml. The solution was vortexed to get a homogenous solution. The stock solution was diluted with HMI-9 culture medium. The diluted fraction/compound solution was filtered into new sterile tubes.

### Trypanosoma Parasites

The GUTat 3.1 strain of the bloodstream forms of T. b. brucei parasites was used for this study. Parasites were cultured in vitro according to the conditions established by Yabu et al, 1998.

Parasites were used when they reached a confluent concentration of 1×10⁶ parasites/ml. Estimation of parasitemia was done with the Neubauer's counting chamber. Parasites were diluted to a concentration of 3×10⁵ parasites/ml with HMI-9 medium and used for the various experiments.

### Determination of Trypanocidal Activity

### Alamar Blue Assay

The Alamar Blue assay was carried out in a 96-well plate. About 1.5×10⁴ Parasites were seeded with varied concentrations of the Morinda lucida crude extract, ranging from as low as 0.78 μg/ml to as high as 200 μg/ml, in a ratio of 1:1 in 100 μl volume per well. Berberine, a compound known as anti-trypanocidal agent, was used as a positive control. Final concentrations of EtOH or DMSO were maintained less than 1% and 0.1%, respectively to ensure they did not have adverse effect on the parasites. The parasites were incubated with the plant extracts for 24 hours at 37° C. and under 5% CO₂. After 24 hours, 10% Alamar Blue dye was added to each well and re-incubated for another 24 hours under the same conditions but in darkness. After a total of 48 hours, the plate was read for absorbance at 540 nm using the TECAN Sunrise Wako Spectrophotometer. An Absorbance-concentration (extract) curve was drawn and the IC₅₀ value of the plant extract was extrapolated. Alamar Blue assay was also performed on the fractions and compounds purified from Morinda Lucida to determine their IC₅₀ values. The IC₅₀ values obtained were compared with that of the positive control and trypanocidal activity determined.

### Apoptosis Induction Assays

These assays were done to investigate the apoptosis-inducing capabilities of all plant materials shown to have trypanocidal activity with very low IC₅₀ values in the Alamar Blue Assay by detecting markers of apoptosis in the parasites.

### Nexin Apoptosis Assay

This assay was performed to detect both Phosphatidylserine (PS) signal and fracture of nuclear membraneas markers of apoptosis. Seeding and incubation of parasites with plant materials (extracts, fractions and compounds) were done under the same conditions in Alamar Blue assay. After 24 hours, Nexin reagent which contains Annexin V-PE and 7-AAD as active ingredients was added to each well in the ratio of 1:1 and incubated for 20 min in darkness after mixing gently with the plate mixer. The contents of the wells were then subjected to FACS analysis using, the Millipore guava easyCyte 5HT FACS machine for the sorting and estimation of apoptotic parasites according to the manufacturer's instructions.

### Multi-Caspase Apoptosis Assay

This assay was performed to investigate multi-caspase signals as markers of apoptosis. Seeding and incubation of parasites were as for the other experiments. After 24 hours of incubation, 10% Caspase Reagent Working Solution was added to each well. The plate was incubated for 1 h at 37° C. under 5% CO₂ in air. The plate was washed twice with 1× apoptosis wash buffer, centrifuged for 5-7 min and the supernatant aspirated off. Two hundred microlitres (200 μl) working solution of Caspase 7-AAD was added to each well and mixed thoroughly to re-suspend parasites. The plate was incubated for 10 min at room temperature and subjected to FACS analysis using, the Millipore guava easyCyte 5HT FACS machine for the sorting and estimation of apoptotic parasites according to the manufacturer's instructions.

### Cell Cycle Assay

This assay was performed to investigate any abnormal change in the parasites' cell cycle which may be caused by extracts/compounds with trypanocidal effect. Five ml of parasites suspension with a concentration of 3×10⁵ cells/ml were incubated with or without anti-Trypanosoma extract/compound at the appropriate IC₅₀ for 24 hours. The parasites were collected and transferred into 15 ml centrifuge tubes and centrifuged at 1700 rpm for 10 min, after which the supernatant was discarded. The pelleted parasites were re-suspended in 5 ml PBS solution and vortexed to form a suspension. The parasite suspension was centrifuged at 1700 rpm for 10 min and the supernatant discarded. The pelleted parasites were re-suspended in 1.5 ml of PBS and vortexed thoroughly before adding 3.5 ml absolute EtOH gradually to a final volume of 5 ml 70% EtOH. The parasites were then fixed at −20° C. for 1 h. The cell suspension was then centrifuged at 1700 rpm for 10 min and the supernatant discarded. Pelleted parasites were re-suspended in 200 μl of Guava Cell Cycle reagent, which has Propidium Iodide as its main constituent and incubated at room temperature for 30 min. The solution was transferred to wells of 96-well plate. The well contents were then subjected to FACS analysis using the Millipore guava easyCyte 5HT FACS machine, for the sorting of parasites according to the manufacturer's instructions.

### Immunohistochemistry

Immunohistochemistry was performed to detect any morphological or phenotypic changes which may be caused by extracts/compounds with trypanocidal effect. The initial steps of immunohistochemistry are the same as that for the Cell Cycle Assay. After fixing the parasites at −20° C. for an hour, 500 μl of the fixed parasites were transferred to an 8-chamber slide. The parasites were incubated at 4° C. overnight to allow the fixed parasites to adhere to the slide surface. The 70% EtOH was then discarded completely. 500 μl of PBS was added to each chamber and incubated for 5 min, after which PBS was discarded. The parasites were further washed with 500 μl of PBST (0.01% Triton X 100 in PBS) for 5 min and the washing solution discarded. 500 μl of blocking reagent (3% BSA in PBST) was added to the chamber and incubated for 30 min at room temperature, after which the blocking reagent was discarded. Each chamber was filled with 500 μl Anti-α tubulin antibody and incubated for 1 hour. The antibody was then discarded and the parasites were incubated with 500 μl of DAPI (5 μg/ml DAPI in PBS) for 10 min in the dark. The wells were washed twice with 500 μl of PBS and once with 500 μl of PBST. The 8-chamber block was detached from the slide and a few drops of parmafluor mounting reagent were put on the slide. The slide was covered carefully with a cover slip. The covered slide was allowed to dry in the dark and the edge of the cover slip sealed with clear nail varnish. The slide was observed under the Olympus fluorescent microscope.

### Results

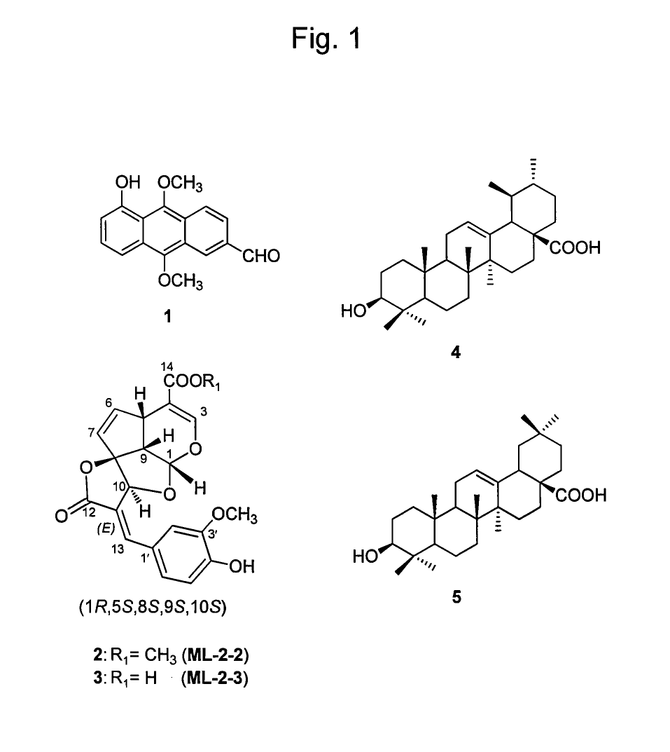

The crude extract of Morinda lucida leaves exhibited a strong inhibition activity on the growth of Trypanosoma brucei brucei. Bioassay-guided fractionation and chromatographic separation of the most active fraction of M. lucida leaves extract resulted in the isolation of five compounds (1-5 below) including two new complex tetracyclic spirolactone iridoids (compounds 2 and 3), together with Oruwalol (1), Ursolic acid (4), and Oleanolic acid (5). Their structures were elucidated based on the extensive spectroscopic methods, and the propriety of structures was confirmed by a biosynthetic pathway hypothesis. Among the isolated constituents, the compound 3 (ML-2-3) showed the most potent in vitro activity against the growth of Trypanosoma cells. FIG. 1 shows the structure of tetracyclic spirolactone iridoids (2: ML-2-2 and 3: ML-2-3, Oruwalol (1), Ursolic acid (4) and Oleanolic acid (5) isolated from leaves of M. lucida. FIG. 2 shows the protocol for fractionating and isolating the compounds.

Compounds 2 and 3 have a rare tetracyclic iridoid skeleton in a molecule. Their biosynthetic pathway hypothesis is unique because they are biosynthesized from iridoid compound and ferulic acid to produce further two rings having a five-membered lactone ring and a five-membered ether ring, and then build up spirolactone-skeleton in a molecule.

Air-dried leaves of Morinda lucida (1100 g) were treated with 50% aqueous EtOH (2.0 L×3 times) at 40° C. and under sonication. After removal of solvent, the crude extract (203 g) was suspended in 1.0 L of water and successively partitioned with hexane, CHCl₃, and EtOAc (each 1.0 L×3) to obtain soluble fractions of hexane (2.10 g), CHCl₃ (3.80 g), and EtOAc (3.6 g). The CHCl₃ fraction, the most active fraction against Trypanosoma cells, was subjected to a silica gel column with hexane-EtOAc (2:1, v/v) as the mobile phase to give seven sub-fractions (fr.1˜fr.7). Fr.4 (120 mg) was further chromatographed over a reversed-phase (RP) column with MeOH—H₂O (3:2, v/v) to yield ML-2-2 (20 mg). Fr.6 (550 mg) was purified by a RP column with MeOH—H₂O (3:5, v/v) followed by a silica gel column (20×350 mm) with CHCl₃-MeOH (25:1, v/v) to give ML-2-3 (50 mg).

Physicochemical and Nuclear Magnetic Resonance (NMR) data of compounds ML-2-2 and ML-2-3 were as following.

Compound ML-2-2:colorless crystal; [α]_{D}²⁵ −188.5° (c1.0, CHCl₃); HR-ESI-MS m/z:

399.1084 [M+H]⁺ (calcd for C₂₁H₁₉O₈, 399.1080); ¹H-NMR (CDCl₃, 400 MHz) δ: 3.58 (1H, dd, J=10.0, 6.0 Hz, H-9), 3.78 (3H, s, 14-COOCH3), 3.96 (3H, s, 3′-OCH₃), 4.05 (1H, dt, J=10.0, 2.0 Hz, H-5), 5.22 (1H, s, H-10), 5.63 (1H, dd, J=6.4, 2.4 Hz, H-7), 5.64 (1H, d, J=5.6, H-1), 6.03 (1H, dd, J=6.4, 2.0 Hz, H-6), 6.99 (11-1, d, J=8.0 Hz, H-5′), 7.26 (1H, dd, J=8.0, 2.0 Hz, H-6′), 7.43 (1H, d, J=2.0 Hz, H-2′), 7.46 (1H, s, H-3), 7.78 (1H, s, H-13); and ¹³C-NMR (CDCl₃, 100 MHz) δ: 102.4 (C-1), 153.0 (C-3), 109.6 (C-4), 38.5 (C-5), 141.1 (C-6), 125.9 (C-7), 104.4 (C-8), 54.3 (C-9), 82.2 (C-10), 120.1 (C-11), 170.0 (C-12), 144.9 (C-13), 166.7 (C-14), 51.7 (14-COOCH₃), 126.5 (C-1′), 112.4 (C-2′), 149.1 (C-3′), 147.0 (C-4′), 115.1 (C-5′), 125.9 (C-6′), 56.0 (3′-OCH₃).

Compound ML-2-3: yellowish powder; [α]_{D}²⁵ −89.2° (c 0.35, CHCl₃); HR-ESI-MS m/z:

385.0925 [M+H]⁺ (calcd for C₂₀H₁₇O₈, 385.0923); ¹H-NMR (CDCl₃, 400 MHz) δ: 3.60 (1H, dd, J=10.0, 6.0 Hz, H-9), 3.95 (3H, s, 3′-OCH₃), 4.05 (1H, dt, J=10.0, 2.0 Hz, H-5), 5.28 (1H, s, H-10), 5.67 (1H, dd, J=6.4, 2.4 Hz, H-7), 5.68 (1H, d, J=5.6, H-1), 6.06 (1H, dd, J=6.4, 2.0 Hz, H-6), 6.92 (1H, d, J=8.0 Hz, H-5′), 7.25 (1H, dd, J=8.0, 2.0 Hz, H-6′), 7.49 (1H, d, J=2.0 Hz, H-2′), 7.50 (1H, s, H-3), 7.75 (1H, s, H-13); and ¹³C-NMR (CDCl₃, 100 MHz) δ: 103.6 (C-1), 153.9 (C-3), 110.2 (C-4), 39.2 (C-5), 141.9 (C-6), 126.9 (C-7), 105.7 (C-8), 54.9 (C-9), 83.0 (C-10), 120.0 (C-11), 169.2 (C-12), 145.9 (C-13), 171.7 (C-14), 127.2 (C-1′), 113.7 (C-2′), 151.1 (C-3′), 148.8 (C-4′), 116.2 (C-5′), 126.0 (C-6′), 56.1 (3′-OCH₃).

From these data, the structures of ML-2-2 and ML-2-3 were elucidated as FIG. 1 shows.

Cytotoxicity of Isolated Compounds from the Leaves of Morinda lucida

The cytotoxic activity of natural products against mammalian cells is an important point in the search for active compounds with biological activity. The results of our investigation demonstrated that the crude extract of Morinda lucida leaves exhibited strong inhibition activity on the growth of T. b. brucei. The results of two new complex compounds, ML-2-2 and ML-2-3, isolated from the CHCl₃ fraction showed the most potent in vitro activity against the growth of T. b. brucei. In order to confirm the selective and potent anti-trypanosomal activity of ML-2-2 and ML-2-3, we examined their cytotoxicity in a panel of human cell lines including two human fibroblastic and 8 human cancer-derived cell lines. Cells were seeded in 96-well microplates and incubated with ML-2-2 and ML-2-3 for 48 h at 37° C. in a 5% CO₂ humidified incubator. The viabilities of cells were determined with the MTT (3-(4,5-dimethylthiazol-2-yl)-2-5-diphenyltetrazolium bromide) assay. The selectivity index (SI) was determined using the following equation: IC₅₀ against human cells/IC₅₀ against T. brucei. The results of cytotoxic activity of ML-2-2 and ML-2-3 are presented in Table 1. The IC₅₀ values of ML-2-3 were higher than those of ML-2-2 in all the cell lines examined. In particular, ML-2-3 was non-toxic at the highest assayed doses in HF-19 and HCT-15. The SI of ML-2-2 indicates values below 1.00 in most cell lines. On the other hand, SI values of ML-2-3 were much higher than those of ML-2-2 in all tested cell lines. These results demonstrated that ML-2-3 could be considered as an excellent lead compound against Trypanosoma.

TABLE 1
Cytotoxicity and SI of ML-2-2 and ML-2-3 against
two human fibroblasts and 8 cancer cell lines

| | | IC50 (μM), 48 h | | | |
|---|---|---|---|---|---|
| | | ML-2-2 | ML-2-3 | SI | |
| IC50 of anti-trypanosomal activity | | 1.27 | 3.75 | ML-2-2 | ML-2-3 |
| Normal fibroblast | NB1RGB | 6.01 | 37.35 | 4.73 | 9.96 |
| | HF-19 | 12.11 | >50 | 9.54 | >13.33 |
| Colon cancer | Caco2 | 1.35 | 44.56 | 1.06 | 11.88 |
| | LoVo | 0.11 | 30.39 | 0.09 | 8.10 |
| | HCT-15 | 16.04 | >50 | 12.63 | >13.33 |
| Stomach cancer | KATOIII | 0.21 | 24.82 | 0.17 | 6.62 |
| Leukemia | Jurkat | 2.45 | 12.41 | 1.93 | 3.31 |
| | U937 | 4.08 | 12.03 | 3.21 | 3.21 |
| | HL-60 | 3.87 | 25.54 | 3.05 | 6.81 |
| | THP-1 | 6.98 | 15.41 | 5.50 | 4.11 |

Anti-Trypanosomal Activities of Isolated Compounds from the Leaves of Morinda lucida

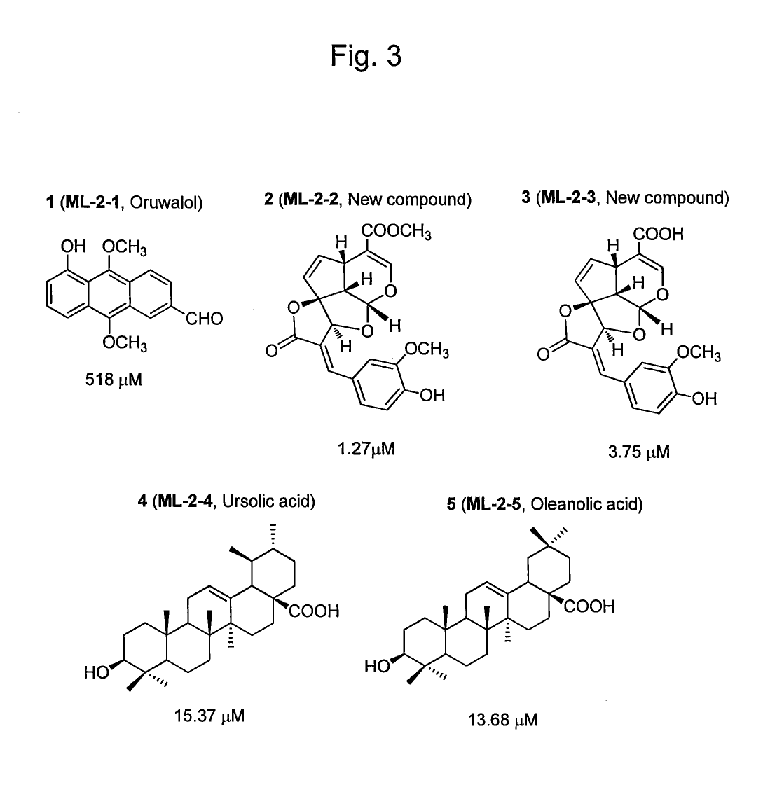

ML-2-2 and ML-2-3 have strong anti-trypanosomal activities with IC₅₀ of 1.27 and 3.75 μM, respectively, compared with other compounds that were isolated from Morinda lucida leaves (FIG. 3).

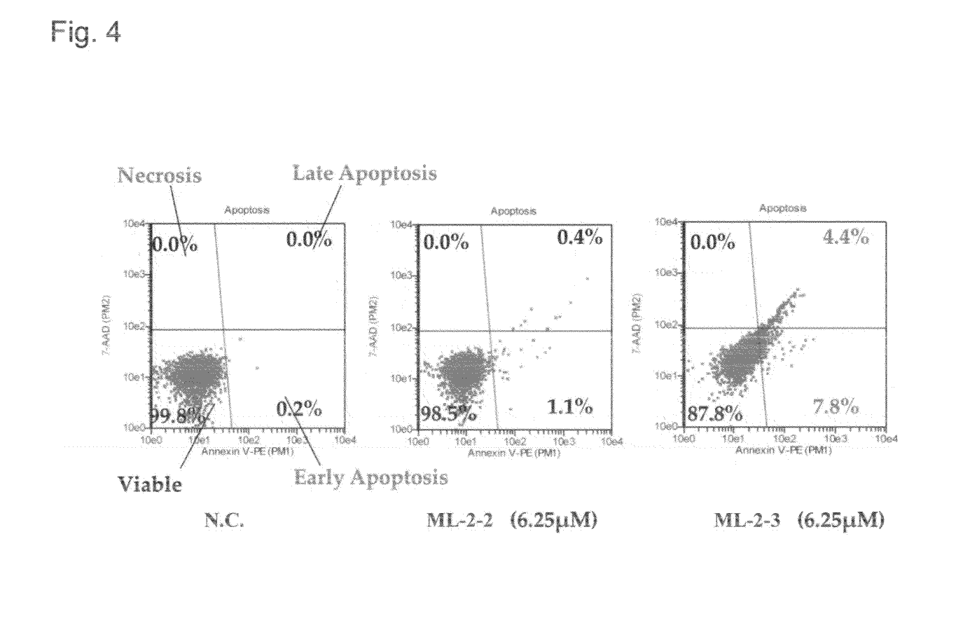

To investigate the mechanism of anti-trypanosomal activities of both ML-2-2 and ML-2-3, apoptosis assay was performed. FACS analysis of ML-2-3-treated Trypanosoma cells subjected to Nexin assay revealed that 6.25 μM of ML-2-3 at 24-hour incubation induced strong apoptosis with 7.8% of early stage and 4.4% of late stages apoptotic cells, whereas treatment-nave Trypanosoma parasites (negative control) showed no late stage and 0.2% of early stage apoptotic cells. On the other hand, 6.25 μM of ML-2-2 showed no significant induction of apoptosis on the parasites even though it has lower IC50 than that of ML-2-3 (FIG. 4). As demonstrated by FIG. 4, ML-2-3-incubated Trypanosoma cells showed significant induction of both early and late stages of apoptosis. N.C. stands for negative control (Trypanosoma cells without incubation with compounds).

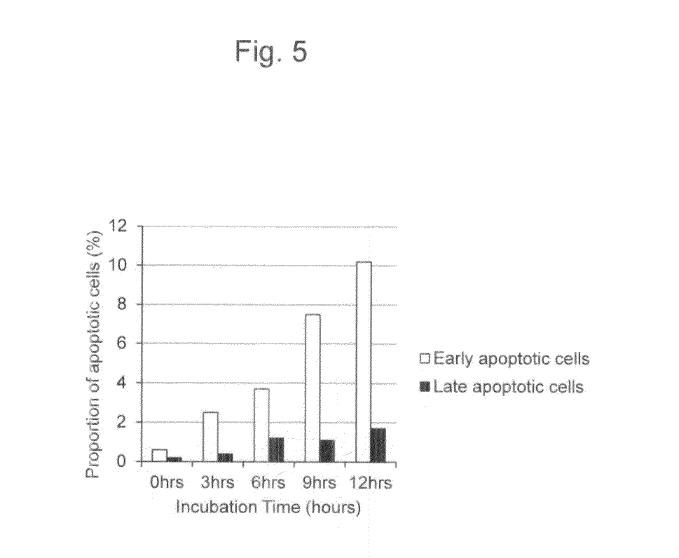

It was found that ML-2-3 but not ML-2-2 induced apoptosis in Trypanosoma cells. The time course of apoptosis induced by ML-2-3 was therefore observed. Trypanosoma cells were incubated for 0, 3, 6, 9 and 12 hours with or without 5 μM of ML-2-3. The proportion of cells at early stage of apoptosis started increasing within 3 hours (2.5%) and reached about 10% at 12 hours of incubation. Significant increase in proportion of late stage apoptotic cells was observed around 6 hours of incubation (FIG. 5).

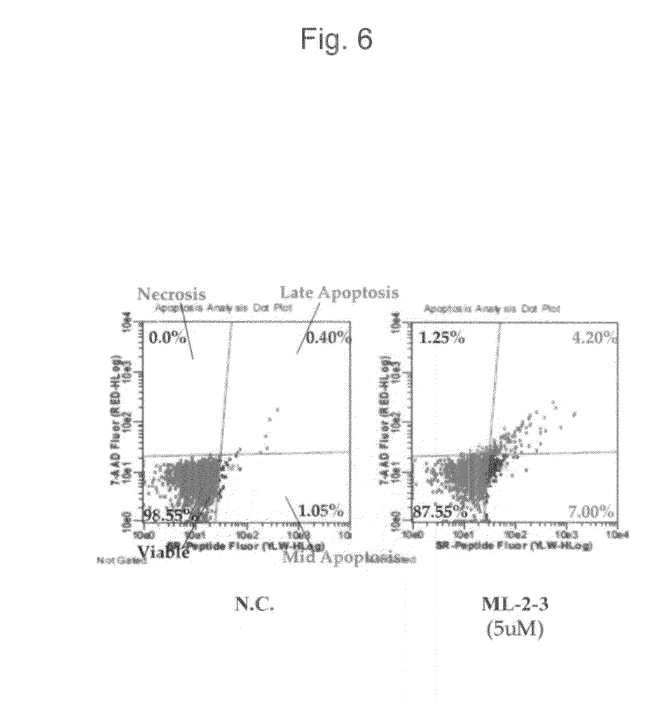

Multi-caspase assay using FACS analysis confirmed that 5 μM of ML-2-3 induced mid-apoptosis in 7% of Trypanosoma cells and late-apoptosis in 4.2% of cells, compared to 1.05% and 0.40%, respectively, in control cells without ML-2-3 treatment (FIG. 6).

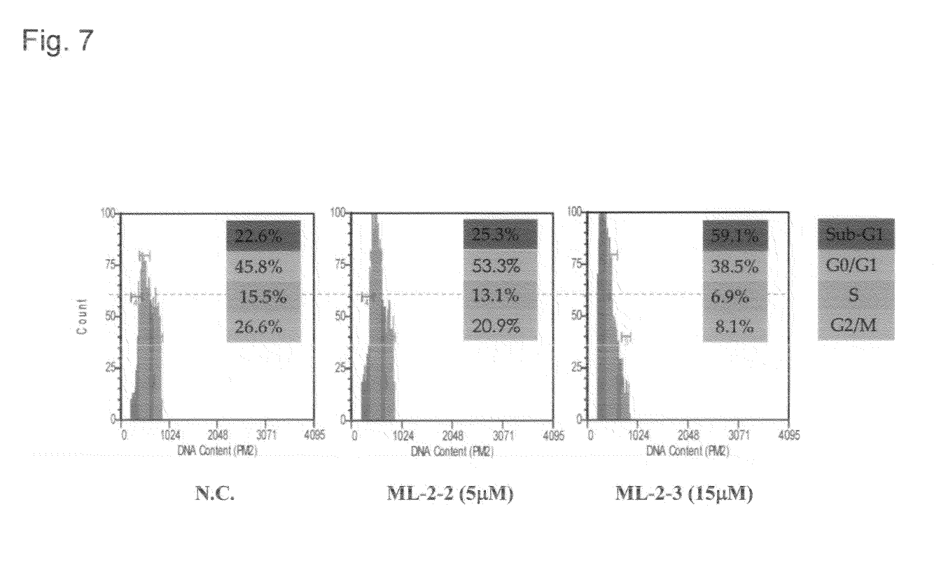

Cell cycle analysis using FACS showed significant decrease in the numbers of cells in the G2 and M phase of cell cycle with 15 μM (four times of IC₅₀) of ML-2-3, whereas 5 μM (four times of IC₅₀) of ML-2-2 did not. In addition, a large proportion of cells with low DNA content were detected as sub-G1 phase with ML-2-3, which indicated DNA fragmentation, a phenomenon found during apoptosis (FIG. 7).

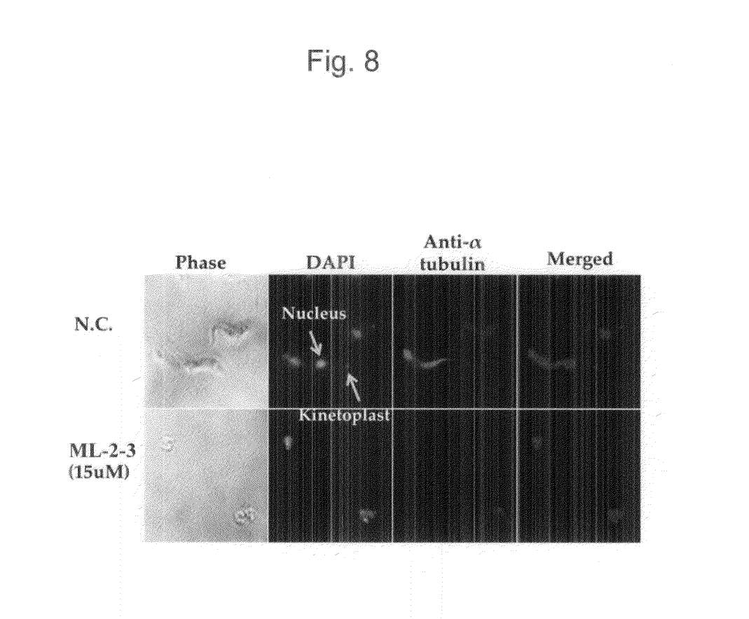

Immuno-fluorescence analysis indicated that the addition of 15 μM (four times of IC₅₀) of ML-2-3 clearly induced the fragmented nuclei in Trypanosoma cells, and the expression of α-tubulin was significantly suppressed, resulting in no flagellum phenotype (FIG. 8).

### Discussion

Two compounds ML-2-2 and ML-2-3 were identified from the extract of leaves of Morinda lucida (ML) with strong trypanocidal activities. The chemical structures showed that their side chains have different functional groups. ML-2-3 has R—COOH that makes ML-2-3 a carboxylic acid whiles ML-2-2 has R—COOCH₃, an ester. These structural differences have associated different physical and chemical properties which include the state of the compound at room temperature, boiling point, melting point, odour and solubility in polar and non-polar solvents. For example ML-2-3 being an acid can undergo hydrogen bonding at the functional group position and will be more soluble in water and other polar solvents relative to ML-2-2. Thin layer chromatography (TLC) results showed ML-2-3 as the more polar compound. These differences between ML-2-2 and ML-2-3 suggested their significant functional differences. In fact, the functional differences between ML-2-2 and ML-2-3 were demonstrated with the mechanistic analysis. It was observed that ML-2-2 was more toxic than ML-2-3 among most of the mammalian cells they were tested. Moreover, ML-2-3 strongly induced apoptosis in Trypanosoma cells whiles ML-2-2 did not at the same concentration (6 μM) although ML-2-2 showed a lower IC₅₀ value (1.27 μM) compared with ML-2-3 (3.75 μM). Furthermore ML-2-3 caused alteration of G2/M phase during cell cycle while ML-2-2 had no effect.

ML-2-2 is stereoisomer of Oruwacin which was reported previously as anti-Leishmania compound extracted from the same plant. Since Leishmania parasites have close relations with Trypanosoma parasites, it is important to ascertain whether Oruwacin and ML-2-2 are functionally the same or not. ML-2-2 and Oruwacin are enantiomers with optical rotations of +193 and −188.5 respectively, in which they have same chemical formula but differ in the arrangement of the atoms in space. The case of enantiomers that have ring forms or bulky side chains such as Oruwacin and ML-2-2 cannot orient easily to the other. Most compounds associate with endogenous receptors or enzymes which are known to be chiral form. These differences eventually lead to a possibility that their function might be different such as one may have anti-trypanosome while the other may have anti-Leishmania properties. An example of a compound with enantiomers having totally different functions is thalidomide which has its R-form inducing sleep and the S-form being teratogenic (Steve R S: Blue Penguin Report: Chirality of Molecules and the Rotation of Polarized Light. http://vagabondgurucom/BluePenguinReportDaily/2009/05/chirality_of_molecules_and_the_rotation_of_polarized_lighthtml 2009: Date retrieved: 24 Oct. 2013).

Interestingly Oruwacin can only be purified within a very short period of a year, three weeks after the rainy season in November in Nigeria, while ML-2-2 can be purified all year round, suggesting that ML-2-2 might be the primary compound of Morinda lucida while Oruwacin is produced from ML-2-2 at certain period of a year.

Although it has not been ascertained if the functional mechanism of Oruwacin against Leishmania and those of ML-2-2 and ML-2-3 against Trypanosoma cells are different, it is important to state if there is any drug available that can treat both pathogens and if its mechanisms are the same. Pentamidine is known to be used to treat both Leishmaniasis and Trypanosomiasis. It also has a long history as treatment for other human protozoan infections such as Babesiosis and Pneumocystis carinii pneumonia (Steve R S: Blue Penguin Report: Chirality of Molecules and the Rotation of Polarized Light. http://vagabondgurucom/BluePenguinReportDaily/2009/05/chirality_of_molecules_and_the_rotation_of_polarized_lighthtml 2009: Date retrieved: 24 Oct. 2013). Moreover, its mode of action revealed that Pentamidine affects not only pathogen but also host cells in nonspecific manner. Pentamidine was shown to directly bind to tRNA with nonspecific manner by hydrophobic and electrostatic interactions, resulting in disruption of tRNA structure and inhibition of its aminoacylation and translation (Sun T, Zhang Y, Nucleic Acids Research 2008, 36(5):1654-1664). Pentamidine is positively absorbed by pathogen and accumulated into their mitochondria and/or kinetoplast. Pentamidine has no effect on nuclear DNA of Trypanosoma cells (Barrett M P et al., British Journal of Pharmacology 2007, 152(8):1155-1171; Wang C C, Annual Review of Pharmacology and Toxicology 1995, 35(11):93-127). In comparison, ML-2-3 in the current study caused fragmentation of nuclear DNA as well as suppressing alpha tubulin expression which resulted in the alteration of flagellum function, indicating that the mode of action of ML-2-3 is different from that of Pentamidine.

Over all, it is concluded from the current findings that ML-2-2, ML-2-3 and Oruwacin are structurally and functionally different from each other. Moreover, the functional mechanism of ML-2-3 might be different from that of Pentamidine.

### INDUSTRIAL APPLICABILITY

The compounds having a tetracyclic iridoid skeleton of the present invention have an excellent anti-trypanosomal activity without cytotoxicity against a patient's cells. The compounds are useful for preventing and treating the diseases caused by Trypanosoma.

All publications, patents, and patent applications cited herein are incorporated herein by reference in their entirety.

1. A compound having the tetracyclic iridoid skeleton represented by a general formula (I). 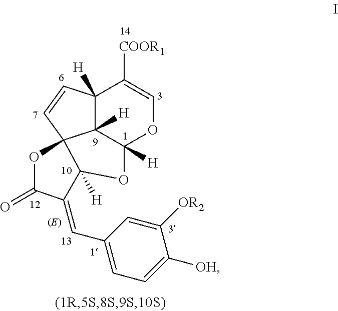
wherein R₁ and R₂ are independently, hydrogen or C₁-C₈ alkyl.

2. The compound according to claim 1, which is isolated from Morinda lucida.

3. The compound according to claim 1, which is represented by a formula (II).

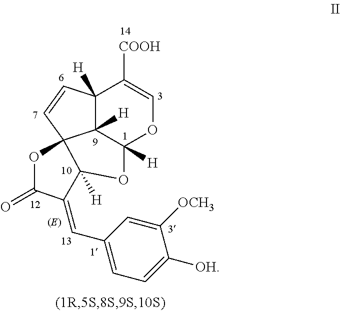

4. The compound according to claim 1, which is represented by a formula (III).

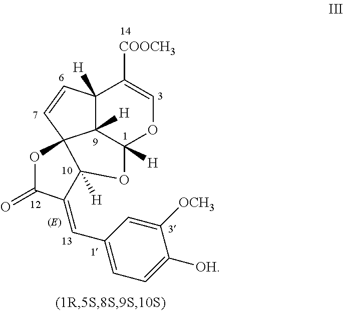

5. An anti-trypanosomal agent for preventing or treating Trypanosomiasis comprising the compound according to claim 1 as an active ingredient.

6. The anti-trypanosomal agent according to claim 5, which comprises the compound according to claim 3 as an active ingredient.

7. The anti-trypanosomal agent according to claim 5, which comprises the compound according to claim 4 as an active ingredient.

8. The anti-trypanosomal agent according to claim 5, wherein the Trypanosomiasis is African Trypanosomiasis or American Trypanosomiasis.

9. The anti-trypanosomal agent according to claim 5, which has low cytotoxicity on mammalian cells.

10. The anti-trypanosomal agent according to claim 5, wherein the selectivity index (SI) of the compound on normal human fibroblast is more than 8.

11. A method for preventing or treating Trypanosomiasis, which comprises administering the compound according to claim 1 to a mammal.

12. The method for preventing or treating Trypanosomiasis according to claim 11, which comprises administering the compound according to claim 3 to a mammal.

13. The method for preventing or treating Trypanosomiasis according to claim 11, which comprises administering the compound according to claim 4 to a mammal.

14. The method according to claim 11, wherein the Trypanosomiasis is African Trypanosomiasis or American Trypanosomiasis.

15. The method according to claim 11, wherein the compound has low cytotoxicity on mammalian cells.

16. The method according to claim 11, wherein the selectivity index (SI) of the compound on normal human fibroblast is more than 8.

17. The method according to claim 11, wherein the subject is a domestic animal.

18. The method according to claim 11, wherein the subject is a human.
